(19) United States
(12) Patent Application Publication
(10) Pub. No.: US 2011/0077444 A1
(43) Pub. Date: Mar. 31, 2011

(54) Metathesis Catalyst for Olefin Production

(75) Inventor: James R. Butler, League City, TX (US)

(73) Assignee: Fina Technology, Inc., Houston, TX (US)

(21) Appl. No.: 12/568,958

(22) Filed: Sep. 29, 2009

(51) Int. Cl. C07C 5/25; B01J 23/28; B01J 23/02

(52) U.S. Cl. 585/670; 502/306

(57) ABSTRACT

A catalyst includes a metathesis catalyst and an isomerization catalyst. In one embodiment, WO₃ is supported on MgO. Alternate embodiments include an inert support, such as alumina or silica, where either one or both of WO₃ and MgO are supported on said inert support. The metathesis catalyst can be used for the metathesis of olefins, such as the metathesis of 2-butene and ethylene to produce propylene.

### CROSS-REFERENCE TO RELATED APPLICATIONS

Not applicable.

### FIELD

The present invention generally relates to catalysts used in the production of propylene via the metathesis of ethylene and C4 hydrocarbons.

### BACKGROUND

Propylene is a short chain mono-unsaturated hydrocarbon that can be produced from steam crackers, fluid catalytic crackers, and other refinery processes. Its chemical formula is C₃H₆. The major use of propylene is in the production of polypropylene, a plastic that can be used in a variety of industries, including automotive, medical, and textile, just to name a few. Propylene can also be used in the production of phenol, acetone and other chemicals. The demand for propylene has generally increased over recent years, which in turn, has increased the need for alternative, or “on-purpose” production of propylene. Such processes include propane dehydrogenation, olefins cracking and interconversion, high severity FCC, and gas-to-olefins.

One common route of propylene production is via the metathesis of 2-butene and ethylene. Metathesis is a general term for a chemical reaction in which chemical moieties are exchanged among a group of molecules. In olefin metathesis, alkylidene moieties are exchanged via the breaking and re-forming of carbon-carbon double bonds. In the production of propylene, the double bond of a 2-butene molecule and the double bond of an ethylene molecule both break and “switch” places. The double bonds reform, yielding two propylene molecules.

The catalyst used for this reaction is generally a transition metal oxide, such as an oxide of molybdenum, rhenium, or tungsten. The feedstock for this metathesis reaction is generally composed of ethylene and a 2-butene source. The 2-butene sources can be either relatively pure 2-butene, or a mixture of 2-butene and its isomer 1-butene, such as that found in a de-isobutanized steam cracker C₄ stream. The metathesis reactor can include an isomerization catalyst to promote the conversion of 1-butene to 2-butene. Magnesium oxide is a common catalyst for this purpose.

When an isomerization catalyst and a metathesis catalyst are used in the same reactor for propylene production, the two metal oxides are often distributed in a layered fashion, wherein the catalyst bed is loaded with alternating layers of metathesis catalyst and isomerization catalyst. When the metathesis catalyst includes tungsten oxide, it often suffers fragility and easy breakdown, which can lead to increased pressure drop across the reactor. Such pressure drop often necessitates regeneration with catalyst removal and screening, which requires undesirable process shutdown. Generally, the pressure drop is decreased by regeneration of the catalyst, but can reoccur more quickly after each regeneration.

Catalyst removal and screening requires process shutdown and can result in the loss of time and money. It is thus desirable to have a tungsten-based metathesis catalyst with enhanced mechanical strength.

### SUMMARY

Embodiments of the present invention generally include a supported catalyst comprising a catalyst for the metathesis of olefins and a catalyst for the isomerization of olefins.

In an embodiment, the metathesis catalyst is supported on the isomerization catalyst.

In another embodiment, the catalyst includes a supported tungsten oxide (WO₃) and magnesium oxide (MgO). The tungsten oxide component catalyzes the metathesis of olefins and the MgO component catalyzes the isomerization of olefins.

In one embodiment, WO₃ is supported on MgO or a MgO based support. A. MgO based support can include an additional inert compound, such as alumina, silica, or a combination thereof. Thus, the MgO component acts both as a support and a catalyst for the isomerization of olefins. The MgO component can catalyze the conversion of terminal olefins, such a 1-butene, to internal olefins, such as 2-butene. When the tungsten catalyst is used for the metathesis of 2-butene and ethylene to produce propylene, the MgO can act as both a support for the catalyst and a catalyst itself, capable of catalyzing the isomerization of any 1-butene present in the reactor to 2-butene.

In an alternate embodiment, the support includes an inert compound, such alumina, silica, or a combination thereof. Both the tungsten oxide and the magnesium oxide components can be attached to the inert support. Alternately, the tungsten oxide component can be attached to the support and then blended with the magnesium oxide component. In these embodiments as well, the magnesium oxide component can act as an isomerization catalyst, when necessary.

In one embodiment, the WO₃ component includes one or more promoters.

In another embodiment, the invention is for a process for the production of olefins that includes the steps of providing a metathesis catalyst having a supported WO₃ and MgO to a metathesis reactor, supplying a feedstock of one or more olefins to the metathesis reactor, contacting the olefin feedstock with the metathesis catalyst within the reactor under conditions effective to produce olefins with a different molecular weight from the feedstock, and recovering a product of olefins with a different molecular weight from the feedstock from the reactor.

The metathesis catalyst can be supported according to any of the above embodiments.

The feedstock can include a mixture of internal and terminal olefins, and the MgO component can catalyze the conversion of terminal olefins to internal olefins. In one embodiment, the feedstock includes a mix of 1-butene and 2-butene, and the MgO catalyzes the isomerization of 1-butene to form 2-butene.

In one embodiment, the feedstock includes 2-butene and ethylene, and the product includes propylene. The feedstock can additionally include 1-butene, which can be isomerized to 2-butene by the MgO component of the catalyst.

In an embodiment, the conditions within the reactor include a temperature of from 100° C. to 600° C. and a pressure of from 0 psig to 200 psig.

### BRIEF DESCRIPTION OF DRAWINGS

FIG. 1 illustrates an embodiment of a reactor scheme for a metathesis reaction over a supported WO₃ catalyst.

### DETAILED DESCRIPTION

The present invention is for a metathesis catalyst that includes tungsten, such as tungsten oxide, and a physical support. The physical support is any that increases the mechanical strength of the tungsten catalyst to prevent breakdown and pressure drop in the metathesis reactor.

In one embodiment, the metathesis catalyst includes a tungsten oxide (WO₃) supported on a magnesium oxide (MgO). In this embodiment, the MgO can act as both a mechanical support and as an isomerization catalyst, for the isomerization of terminal olefins such as 1-butene to internal olefins such as 2-butene. Terminal olefins have a carbon-carbon double bond positioned at the end, or alpha position, of a hydrocarbon chain, whereas internal olefins have a carbon-carbon double bond positioned inside the chain, starting at the second or higher carbon atom. The magnesium oxide support can optionally include an amount of some inert support. For example the magnesium oxide support can be a co-gel of magnesium and alumina or silica. The catalyst can range from 5 wt % to 100 wt % MgO support and from 1 wt % to 50 wt % WO₃. Optionally the catalyst can range from 10 wt % to 90 wt % MgO. Optionally the catalyst can range from 5 wt % to 40 wt % WO₃.

In another embodiment, the metathesis catalyst includes a tungsten oxide, a magnesium oxide, and an inert support. The tungsten and magnesium can either be attached, such as by impregnation, simultaneously to the support, or separately. During a calcination step the tungsten can oxidize to tungsten oxide and the magnesium can oxidize to magnesium oxide. Further, the tungsten only can be impregnated to the support, calcined and then blended with magnesium oxide. Other configurations are possible, and any configuration in which an inert support enhances the mechanical strength of the metathesis catalyst is suitable to the present invention. The inert support can be any known in the art, generally a porous mineral substrate, that does not interfere with or materially affect the metathesis and isomerization reactions taking place in the metathesis reactor. Examples of supports that can potentially be used in the present invention include alumina-based supports and silica-based supports. Other ceramic supports, such as titanium and zirconium; oxides or phosphates of silica, alumina, titanium, thorium, zinc, botium and zirconium; magnesia, clays, cements, and similar species may also be used. Combinations of any of the listed supports can also be used, as well as any of the listed supports that are charged with a promoter. The catalyst can range from 0.1 to 40 weight percent tungsten metal, optionally 1 to 15 weight percent.

In either embodiment, the magnesium and tungsten used in the preparation of the catalyst can be either oxides or precursors that become oxides during such treatments as calcination. Suitable magnesium precursors can include oxides, hydroxides, nitrates, sulphates, acetates, and their mixtures. Furthermore, magnesia can be naturally occurring, such as the mineral brucite, or can be synthetically prepared by suitable techniques. Suitable tungsten precursors can include oxides, halides, sulphides, sulphates, nitrates, acetates and their mixtures. Some specific tungsten compounds that can be used include tungsten pentachloride, tungsten dichloride, tungsten tetrachloride, tungsten hexafluoride, tungsten trioxide, tungsten dioxychloride, tungsten trisulphide, metatungsten acid, orthotungsten acid, ammonium phosphotungstenite and ammonium metatungstenite. Tungsten-based catalysts can also include a promoter to enhance performance. Such promoters are known in the art and can be selected from among such species as alkali metals, phosphates, borates, cobalt oxide, and can include inorganic bases such as NaOH, KOH, and the like.

The catalyst can be prepared according to any known procedure in the art wherein a support is charged with a metal oxide, including such procedures as co-precipitation, dry mixing, and impregnation. After preparing the catalyst, it can be dried and calcined. Drying can take place at temperatures from 50° C. to 250° C. Calcination can take place in the presence of an oxygen-containing gas, such as air, at a temperature of from about 300° C. to about 800° C. for about 10 minutes to 20 hours. After calcination, the catalyst can be flushed with an inert gas such as nitrogen or argon. The catalyst can then be shaped or meshed according to the reactor type and the desired physical form of the catalyst. Suitable physical forms can include powders, irregular chunks, beads, pellets, extrudates, agglomerates, granules, spheres, balls, and the like. In an embodiment wherein tungsten is not deposited on a magnesium support nor are magnesium and tungsten co-deposited on a shared inert support, the magnesium oxide and the tungsten oxide can be intimately blended such as by grinding and the powder then formed into other shapes such as pellets, tablets, agglomerates, extrudates, and the like, such that the catalyst in the catalyst bed is an intimate blend of the two oxides.

The present invention in its many embodiments can be used in many different reactions, and is suitable for the production of propylene via the metathesis of 2-butene and ethylene. The reaction conditions can include those which are known in the art as being effective for the metathesis reaction. For example, the temperature can be from 100° C. to 600° C., optionally from 200° C. to 500° C., optionally from 300° C. to 400° C.; the pressure can be from 0 psig to 2000 psig or more, optionally from 200 psig to 1000 psig optionally from 300 psig to 500 psig; and the weight hourly space velocity from 1 h⁻¹ to 100 h⁻¹, optionally from 2 h⁻¹ to 75 hr⁻¹, optionally from 5 h⁻¹ to 30 hr⁻¹. Many different reactor types can be used with the catalyst of the present invention. Metathesis reactions can be carried out in either a gas or a liquid phase.

The feedstock generally includes ethylene and a source for 2-butene. The source for 2-butene can be relatively pure 2-butene, or can be a mixture of 2-butene and its isomer 1-butene. Both the cis and trans isomers of 2-butene are suitable for the metathesis reaction with ethylene. The 2-butene source can include inerts, such as butane and iso-butane, that do not interfere with the reaction. Contaminants that do interfere with the reaction, including other butene isomers, C4 olefins, dienes, oxygen, water, sulfur, etc. can, and generally must, be removed from the butene stream prior to its introduction into the metathesis reactor. Such procedures, such as distillation, are well known in the art.

Although the catalyst is predominately described in reference to the metathesis of 2-butene and ethylene to produce propylene, the catalyst can be used for many metathesis reactions. Suitable reactants include mono-olefins having 2 to 30 carbon atoms, such as ethylene, propylene, butenes, pentenes, hexenes, and octenes; cyclic mono-olefins having 3 to 20 carbon atoms, such as cyclopentene, cyclooctene, and norbornene; poly-olefins having 4 to 30 carbon atoms, including dienes such as hexadiene-1,4 and octadiene-1,7; and cyclic polyolefins having 5 to 30 carbon atoms, such as cyclooctadiene-1,5, norbornadiene, and dicyclopentadiene. Olefins falling into any of the aforementioned categories can optionally carry functional groups, such as halogens, ethers, nitriles, amines, amides, and silanes or ester groups, such as methyl oleate. Possible metathesis reactions include “self-disproportionation” reactions wherein a single olefin is used as the reactant, and “co-metathesis” reactions wherein two or more different olefins are used as the reactants.

In its many embodiments, the metathesis catalyst can be described in terms of the proportion of its metathesis catalyzing component, or WO₃, to its isomerization catalyzing component, or MgO. Such proportion can be varied spatially throughout the reactor. For example, one reactor scheme can include a greater relative amount of MgO upstream for a high butane-1 feed and an equal or greater relative amount of WO₃ downstream, such that isomerization takes places predominately upstream and metathesis takes place predominately downstream. Alternate arrangements are also possible and can be determined by one skilled in the art.

An embodiment is a method for the production of olefins via the use of a supported metathesis catalyst such as WO₃. The metathesis catalyst can be prepared in accordance with any of the above described embodiments. The method includes providing a metathesis reactor with a supported metathesis catalyst and an isomerization catalyst, providing reactants to the reactor inlet, reacting the reactants over the catalysts to produce a product, and collecting the product at the reactor outlet. In an embodiment, the reactants include a 2-butene source and ethylene, and the product consists primarily of propylene. Products leaving the reactor can be treated according to various downstream processes. For example, the desired product can be isolated via distillation, and any unreacted feedstock can be recycled to the metathesis reactor.

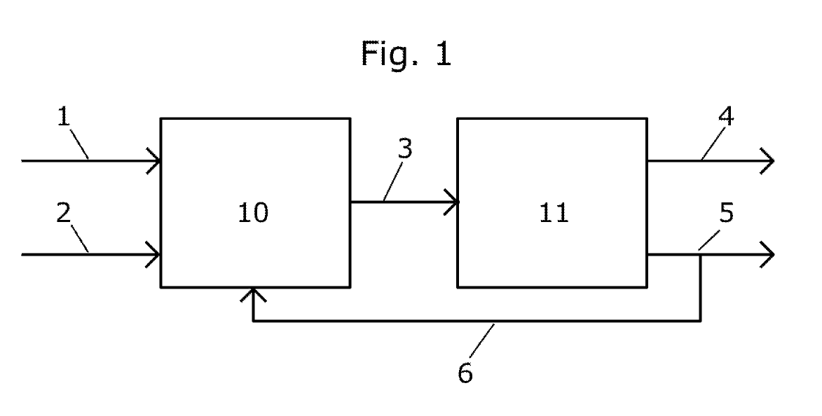

FIG. 1 shows an embodiment of the present invention. A reactor 10 is supplied with a supported WO₃ metathesis catalyst and a MgO isomerization catalyst. The WO₃ can be supported on the MgO, or optionally can be supported on an inert support. The reactor 10 is fed ethylene via line 1 and a 2-butene source via line 2. The 2-butene source can include relatively pure 2-butene, or optionally a mixture of 1-butene and 2-butene. In the latter case, 1-butene can be converted to 2-butene in the reactor 10 by the MgO catalyst. In either case, 2-butene and ethylene can react over the metathesis catalyst WO₃ to form propylene. The propylene product plus any side products and/or unreacted feedstock can leave the reactor 10 via line 3. Line 3 proceeds to a unit 11 for the separation of propylene from other products. Unit 11 can be, for example, a distillation column. Product propylene can leave unit 11 via line 4. Side products such as pentene-2 can leave unit 11 via line 5. Unreacted feedstock can be recycled via line 6 back to the reactor 10 to undergo metathesis. FIG. 1 is meant to provide a better understanding of an embodiment of the present invention, and several reaction schemes are possible without departing from the scope of the present invention. For instance, various reactor types and forms of both upstream and downstream processing can be used in the present invention and can be readily determined by one skilled in the art.

The term “metathesis” refers to a rearrangement or exchange of chemical moieties among one or more chemicals. For the purposes of the present invention, the term “metathesis” generally refers to the metathesis of olefins, wherein alkylidene moieties are exchanged via the breaking and re-forming of carbon-carbon double bonds.

The term “olefin” refers to any unsaturated chemical compound with at least one carbon-carbon double bond. For the purposes of the present invention, the term “olefin” generally refers to a hydrocarbon with at least one carbon-carbon double bond that is either cyclic or straight-chain. A “mono-olefin” possesses only one carbon-carbon double bond, while a “poly-olefin” possesses two or more carbon-carbon double bonds.

The term “support” refers to any physical support to which the catalyst is bound and which provides the catalyst with enhanced stability and mechanical strength.

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Other and further embodiments, versions and examples of the invention may be devised without departing from the basic scope thereof and the scope thereof is determined by the claims that follow.

What is claimed is:

1. A supported catalyst comprising a metathesis catalyst for the metathesis of olefins and a isomerization catalyst for the isomerization of olefins.

2. The catalyst of claim 1, wherein the metathesis catalyst is supported on the isomerization catalyst.

3. The catalyst of claim 2, wherein the metathesis catalyst comprises WO₃ and the isomerization catalyst comprises MgO.

4. A supported tungsten catalyst, used for the metathesis of olefins, comprising WO₃ and MgO.

5. The catalyst of claim 4, wherein the WO₃ is supported on a support comprising MgO.

6. The catalyst of claim 5, wherein the support further comprises an inert support material comprising alumina, silica, or a combination thereof.

7. The catalyst of claim 4, further comprising an inert support.

8. The catalyst of claim 7, wherein the inert support comprises alumina, silica, or a combination thereof.

9. The catalyst of claim 7, wherein WO₃ and MgO are both attached to the inert support.

10. The catalyst of claim 7, wherein WO₃ is attached to the inert support and then blended with the MgO.

11. The catalyst of claim 4, further comprising a promoter.

12. The catalyst of claim 4, wherein the olefins comprise ethylene, 1-butene, and 2-butene.

13. The catalyst of claim 12, wherein the MgO catalyzes the isomerization of 1-butene to 2-butene.

14. The catalyst of claim 12, wherein the WO₃ catalyzes the metathesis of 2-butene and ethylene to propylene.

15. A process for the production of olefins comprising:
providing a metathesis catalyst comprised of a supported WO₃ and MgO to a metathesis reactor;
supplying a feedstock comprised of one or more olefins to the metathesis reactor;
contacting the feedstock with the metathesis catalyst within the metathesis reactor under conditions effective to produce olefins with a different molecular weight from the feedstock; and
recovering a product of olefins with a different molecular weight from the feedstock from the reactor.

16. The process of claim 15, wherein WO₃ is supported on a MgO support.

17. The process of claim 16, wherein the MgO support further comprises an inert support material comprising alumina, silica, or a combination thereof.

18. The process of claim 15, wherein WO₃ and MgO are both supported on an inert support.

19. The process of claim 18, wherein the inert support comprises alumina, silica, or a combination thereof.

20. The process of claim 15, wherein the WO₃ is supported on an inert support and blended with the MgO.

21. The process of claim 15, wherein the feedstock comprises a mix of terminal and internal olefins.

22. The process of claim 21, wherein the MgO catalyzes the isomerization of terminal olefins to internal olefins.

23. The process of claim 15, wherein the feedstock comprises a mix of 1-butene and 2-butene, and the MgO catalyzes the isomerization of 1-butene to form 2-butene.

24. The process of claim 23, wherein the MgO catalyzes the isomerization of 1-butene to form 2-butene.

25. The process of claim 23, wherein the feedstock further comprises C₄ saturated hydrocarbons.

26. The process of claim 21, wherein the feedstock further comprises ethylene.

27. The process of claim 26, wherein the WO₃ catalyzes the metathesis of 2-butene and ethylene to propylene

28. The process of claim 15, wherein the metathesis catalyst comprises a promoter.

29. The process of claim 15, wherein the conditions include a temperature of from 100° C. to 600° C. and a pressure of from 0 psig to 200 psig.
